(19) United States
(12) Patent Application Publication
(10) Pub. No.: US 2008/0123054 A1
(43) Pub. Date: May 29, 2008

(54) Phoropter aligner

(76) Inventor: James F. Humber, Clarksdale, MS (US)

Correspondence Address: James F. Humber, IV, 508 Colonial Drive, Clarksdale, MS, 38614, US

(21) Appl. No.: 11/414,981

(22) Filed: Jul. 24, 2006

(51) Int. Cl. A61B 3/032

(52) U.S. Cl. 351/237

(57) ABSTRACT

A phoropter aligner is disclosed. The device is attached to a phoropter. This device will allow an eyecare practitioner to align the phoropter correctly and be assured that the letters on the visual acuity chart are the correct size. The device will accomplish this by projecting an image from the phoropter onto the center of a visual acuity chart.

### DESCRIPTION

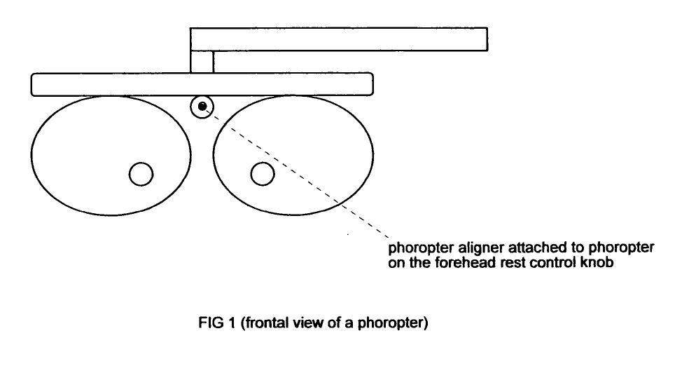

This patent is for a laser or image projecting device known as a phoropter aligner that is attached to the phoropter (FIG. 1). A phoropter is an instrument commonly used by optometrists and ophthalmologists during an eye examination to determine an individual's eyeglass prescription. Typically, the patient sits behind the phoropter, and looks through it at an eye chart. This device (phoropter aligner) projects an image to the eye chart. The size of this image will allow the user to calibrate the size of the letters on the eye chart.

For example, the laser could project a standard size 20/400 letter to a chart that is 20 feet away. The chart projector could then be adjusted so that the size of the 20/400 letter it projects corresponds to the standard laser projected 20/400 letter. When the laser projected letter is centered on the chart, the examiner knows that the phoropter is aligned with the chart. This procedure could be applied to eye charts of all distances, ensuring that the size of the letters on the chart is correct for that distance. Also, this will eliminate errors in determining the refractive error (prescription) of a patient in which the phoropter is out of alignment.

What is claimed is:

1. A device will align a phoropter and ensure that the letters on the visual acuity chart are properly calibrated. It will do this by projecting an image from the phoropter onto the visual acuity chart with the use of a laser or other image producing device.
